(19) United States
(12) Patent Application Publication
(10) Pub. No.: US 2021/0142880 A1
(43) Pub. Date: May 13, 2021

(54) Method for real-time processing and presentation of audio signals for reducing sound hypersensitivity.

(71) Applicants: Nitin Bhalchandra Bangera, Santa Monica, CA (US); Jeffrey David Lewine, Corrales, NM (US)

(72) Inventors: Nitin Bhalchandra Bangera, Santa Monica, CA (US); Jeffrey David Lewine, Corrales, NM (US)

(21) Appl. No.: 17/092,190

(22) Filed: Nov. 6, 2020

Related U.S. Application Data
(60) Provisional application No. 62/933,360, filed on Nov. 8, 2019.

(51) Int. Cl. G16H 20/30; A61M 21/00; H04R 3/00

(52) U.S. Cl. CPC G16H 20/30; A61M 21/00; A61M 2021/0055; A61M 2021/0027; H04R 3/002

(57) ABSTRACT

One or more embodiments of the present invention relate to implementation of a music-based strategy for reducing sound hypersensitivity and phonophobia. The method involves presentation of music modified through amplitude and frequency filtering based on individual audiometric profiles (including information on auditory thresholds and uncomfortable loudness levels), and with insertion of additional sounds designed to trigger and guide neuroplasticity. This modified music is presented over a specific duration (two 30-minute sessions per day, 5 days per week for 4 weeks) for the reduction of sound hypersensitivities. For the purposes of this invention, this music processing method applied over a specific duration (40 half hour sessions) is referred to as Advanced Auditory Processing Training (adAPT).

### CROSS-REFERENCE TO RELATED APPLICATION

This application claims the benefit of U.S. Provisional Patent Application No. 62/933,360, filed Nov. 8, 2019, from which priority is claimed under 35 USC § 119(e), and which provisional patent is incorporated by reference herein in its entirety.

### STATEMENT REGARDING FEDERALLY SPONSORED RESEARCH

This invention was made with government support under grant award 7R01 HD0551747 from the National Institute of Child Health and Development; and award 1 R41 DC013197 from the National Institute on Deafness and other Communication Disorders. The government may have certain rights in the invention.

### REFERENCES

Adrien J L, Ornitz E, Barthelemy C, et al.: (1987) The presence or absence of certain behaviors associated with infantile autism in severely retarded autistic and non-autistic retarded children and very young normal children, J Autism Dev Disord 17: 407-416.
Baranek G T, Carlson M, Sideris J, et al.: (2019) Longitudinal assessment of stability of sensory features in children with autism spectrum disorder or other developmental disorders. Autism Res, 12(1):100-111.
Berard G: (1993), Hearing Equals Behavior (Trans.), Keats, New Canaan Conn., [original work published 1982].
Cheung P P, Siu A M. (2009) A comparison of patterns of sensory processing in children with and without developmental disabilities. Res Dev Disabil., November-December; 30(6):1468-80. doi: 10.1016/j.ridd.2009.07.009.
Delacato, C. H. (1974) The ultimate stranger: The autistic child. Oxford, England: Doubleday.
Dickie V A, et al.: (2009) Parent reports of sensory experiences of preschool children with and without autism: a qualitative study, Am J Occup Ther, March-April; 63(2):172-81.
DiLelia D L, Rogers S J: (1994) Domains of the Childhood Autism Rating Scale: relevance for diagnosis and treatment, J Autism Dev Disord 24(2):115-28.
Dunn W, Saiter J., & Rinner, L. (2002) Asperger Syndrome and Sensory Processing: a conceptual model and guidance for intervention planning, Focus on Autism and other Developmental Disabilities, 17(3), 172-185.
Frith U and Barron-Cohen S: (1987) Perception in Autistic Children. In: D Cohen, A Donnellan and R Paul (eds.), Handbook of Autism and Disorders of Atypical Development, pp 85-102, New York, Wiley Press.
Hanson E, et al: (2007) Use of complementary and alternative medicine among children diagnosed with autism spectrum disorder, J Autism Dev Disord, 37(4):628-36.
Khalfa S et al.: (2004) Increased perception of loudness in autism, Hear Res., 198(1-2):87-92.
Rogers S J, Ozonoff S: (2005) What do we know about sensory dysfunction in autism? A critical review of the empirical evidence, J Child Psychol Psychiatry, 46(12):1255-68.
Sinha Y, Silove N, Wheeler D, et al: (2006) Auditory integration training and other sound therapies for autism spectrum disorders: a systematic review, Arch Dis Child, 91(12):1018-22.
Tomchek S D, Dunn W.: (2007) Sensory processing in children with and without autism: a comparative study using the short sensory profile. Am J Occup Ther, March-April; 61(2):190-200.

### BACKGROUND OF THE INVENTION

### Field of the Invention

The present invention relates to a method for processing of audio signals in real time and the specific combination of processing steps of the audio signal over a specific period of time for reducing sound hypersensitivities. Specific processing parameters for each individual client may be based on that individual's audiometric profile. The invention also relates to a computer program for real time implementation of the method of processing the audio signal.

### Discussion of State of the Art

The Autism Spectrum Disorders (ASDs) are generally considered to be some of the most serious of the neuro-developmental disorders, with recent CDC reports suggesting an autism epidemic. The current prevalence rate of ASDs in the United States is 1%, with available data indicating that this rate is increasing by ˜10% per year (see www.cdc.gov/ncbddd/autism/index.html). Although sensory processing problems are not considered to be core diagnostic symptoms of autism, both clinical observations (Adrien et al., 1987; DiLalla and Rogers, 1994) and parental questionnaires (e.g., Cheung et al., 2009; Tomchek and Dunn, 2007; Dunn et al., 2002) confirm the presence of sensory anomalies in 42-88% of school age children with autism (see Dickie et al., 2009). Sound sensitivities seems to be especially problematic, with 25-30% of autistic children showing extreme discomfort when they hear loud sounds such as those associated with a vacuum cleaner, washing machine, baby crying, fireworks, thunder, etc. (Delacato, 1974; Frith and Baron-Cohen, 1987; Khalfa et al., 2004). Remediation of sound hypersensitivities is an important therapeutic target because there is mounting evidence that auditory processing problems contribute directly to behavioral irritability and impaired language/social-communication skills (Rogers and Ozonoff, 2005).

Although the basic biology of sound sensitivities in autism is not well characterized, there are numerous anecdotal reports that some music-based therapies can alleviate sound sensitivities and provide improvement in autistic features (e.g., increased communication, reduced aberrant behaviors). Relevant therapies include (1) the Tomatis method, (2) The Listening Program (TLP), and (3) Berard Auditory Integration Training (AIT). While upwards of 50% of children with autism might benefit from auditory therapy, only 2-4% actually receive this type of intervention (Hanson et al., 2007). Several factors limit the widespread use and acceptance of music-based therapies in autism. First, there are only very limited data actually documenting effectiveness. The best studied method is Berard AIT (Berard, 1993). Berard AIT involves listening to 10 hours of modulated music that is often subjected to additional narrow band filtering (based on auditory thresholds). While there have been a handful of studies showing positive benefit for AIT, other studies have failed at demonstrating efficacy (Sinha et al., 2006 for a review), and there is no neurobiological data supporting proposed mode-of-action for Berard's AIT. The other methods have essentially no scientific documentation. Another limiting factor is that the proposed main mode of action for these methods relates to unsubstantiated claims of dysfunction of the muscles of the middle-ear, with therapy postulated to ‘exercise and fine tune’ the middle ear. A third factor is the high cost and inconvenience of implementation. TLP, the only program with a parent-guided home implementation plan, typically costs between $500-$1500 (dependent upon the inclusion of a bone-conduction aspect of the therapy). The Tomatis method has a typical cost in excess of $3000, and it must be done in a professional's office or with the therapist coming to the home. Berard AIT costs between $1000-$2000 and is done as an office-based therapy or the therapist coming to the home with the equipment, involving two daily visits (with a minimum of 3 hours between visits) for 10-days. Thus, these aforementioned therapies are inconvenient, expensive and the scientific basis of the mode of action are unclear.

Clearly, the development of a more effective, valid, and inexpensive method of auditory remediation is desirable.

### BRIEF SUMMARY OF THE INVENTION

The results of a series of psychophysical and neuroimaging studies on the biology of sound sensitivities in children with autism (as conducted by the inventors of this invention and colleagues with funding from the NIH, Cure Autism Now, and the Wallace Foundation) have led to a new theory about the basis of sound sensitivities (related to cortical disorganization), and have led to the development of an alternative remediation strategy for sound hypersensitivities. as described in this invention. The purpose of the methods described in this invention is to process audio data such as music to generate a modified version of the music in real-time that is suitable for therapy of sound hypersensitivity. Another object of this invention is the presentation of a specific combination of the processing steps for each of two 30-minute sessions per day for a total of 20 days (5 days a week for 4 weeks) that is suitable for therapy of sound hypersensitivity. Unlike Berard AIT, the pattern of music processing in the described invention is guided by neurobiological data. Unlike other therapies, our method includes (1) independent right and left ear modulation; (2) individualized filtering profiles based on measures of suprathreshold uncomfortable loudness levels (that is, filters are based on auditory tests which evaluate how loud a sound can be before it is perceived as uncomfortably loud) rather than perceptual thresholds (filters are chosen based on peaks and valleys in audiograms that measure the softest sound that can be heard); and (3) inclusion of additional sounds designed to habituate auditory cortex to novel transients. Another major advantage of this invention is the low-cost of implementation (less than US $50) as the music modulation is done in real time via an application on a mobile device (such as smartphones, tablets) for use at home. This makes the therapy easy, affordable, and convenient for families.

The input audio signal is a digital audio signal with two channels which contains digital audio samples. The height of the audio signal gives the amplitude of the audio at a sample of time. The audio signal is characterized by the sampling frequency and the sampling bit. The sampling frequency gives the number of samples per unit of time and the sampling bit gives the resolution of each sample of the audio signal. The audio signal (music) is chosen to have strong low, mid, and high frequency components, including voice. The audio signal may be available in a lossless format (e.g. way) or a lossy and compressed format (e.g. mp3). When the digital audio is encoded in a compressed format, the signal is decoded before processing. When multiple songs are processed, the audio signals are normalized before the processing steps. In this case, normalization refers to scaling the audio samples of each audio signal such that the overall loudness of an audio file is equal to a common reference level. The processing of the digital audio signals is carried out on a data processing device that is available commonly such as a smartphone, mobile tablet or a laptop. The digital audio signals are saved locally on the data processing device as a digital audio file. This may be achieved by loading the digital audio file to the data processing device with a direct connection such as an external hard drive or it is transmitted to the data processing device via the internet from an external server. When the files are saved locally, the entire input audio signal is available to be read in its entirety. The digital audio file may also be available to the data processing device from a music streaming service (such as Spotify or Apple Music) where consecutive segments of the audio signal are transmitted to the data processing device in real time and the whole audio file is not available to the data processing device in its entirety. A data playback device converts the digital output audio file to an analog signal and plays the processed audio via headphones or earphones. In the case of portable data processing devices such as smartphones, tablets or laptops, the data processing device is same as the data playback device.

The methods as described by the invention are executed by a computer code (such as an application/app on a mobile device or program on a laptop) on the data processing device which is also the data playback device. The methods as described by the invention can also be alternatively executed by electronic components in conjunction with the audio data playback device. In such scenario, the data processing device is the electronic component (for example, hardware digital signal processing with microelectronic components) and is different from the audio data playback device.

For the purposes of reducing sound hypersensitivity, multiple input audio signals (digital audio files) with a total duration of approximately 30 minutes are processed and presented to the individual via the data playback device. For the purposes of this invention, we refer to this 30-minute duration as a session. Auditory processing benefits are expected after a completion of 40 sessions. Sessions are split across 4 weeks, with 5 days per week and two sessions per day. The processing of the audio signal for therapy of sound sensitivity based on individual uncomfortable loudness level uses a combination of one of more of the three steps that are referred to as the Neuroplasticity Conditioning (NC), Auditory Shaping (AS) and Habituation Sound Addition (HSA) respectively. A specific combination of the three audio processing steps is used for each of the 40 sessions for purpose of therapy of sound hypersensitivities in an individual. For the purposes of this invention, listening to music modified by a specific combination of the three processing steps for each of the 40 session over 4 weeks is referred to as advanced Auditory Processing Training (adAPT).

The objective of Neuroplasticity Conditioning (NC), the first step of processing is to intentionally produce a very abnormal pattern of cortical stimulation, a pattern that helps to trigger brain plasticity and reorganization of the auditory cortex. In this step, the music is subjected to short-duration high frequency band pass filtering, mid frequency band stop filtering, or low frequency band pass filtering, applied separately to right and left ears. This is achieved by picking randomly from one of the three pre-defined filters for every 0.5 second segment of audio signal and filtering out the frequencies using the randomly picked filters. The three pre-defined filters are a high-pass filter, band stop filter and a low-pass filter respectively that block out frequencies from the first audio signal in the low frequency band (0-500 Hz), mid-frequency (500-4000 Hz) and high frequency band (4000-20000 Hz) respectively. These three frequency bands cover the audio spectrum range for human hearing. The high pass filter blocks frequencies lower than the cutoff frequency (500 Hz) and permits signals with frequencies higher than the cutoff frequency from the input audio signal. The low pass filter allows frequencies lower than a cutoff frequency (3500 Hz) and blocks signals with frequencies higher than the cutoff frequency from the input audio signal. The band stop filters blocks frequencies in a given frequency range (500-3500 Hz) from the input audio signal. For a 2-channel audio signal, the filter assignment is also randomized between the 2 channels (left and right ear).

During Auditory Shaping (AS), additional narrow-band filtering is applied to the music modified by NC. In this step, narrow-band filters are individually set based on psychophysical evaluation of uncomfortable loudness levels (UCLs). Uncomfortable loudness levels (UCLs) are determined through a hearing test which identifies the level (intensity of sound) at which an individual reports sound to be uncomfortably loud. For identification of UCLs, the audio spectrum range between 20 Hz to 20000 Hz can be divided into 11 octave bands centered around 11 frequencies. For finding the Uncomfortable loudness level (UCL), the individual is tested by presenting tones at each of the 11 frequencies. The sound intensity at which discomfort is experienced by an individual for a particular frequency is marked as the UCL for that frequency. UCLs below 90 db are seen in individuals with sound hypersensitivities. Frequencies at which the UCL is below 90 dB are used for Auditory Shaping. The second audio signal is filtered using band-stop filters at frequencies at which UCL is below 90 dB (sound sensitive). In the scenario where the uncomfortable loudness levels are not available, the filters are selected from the group of pre-selected band-stop filters, with pseudo-random application of band-stop filters across sessions.

Finally, in the Habituation Sound Addition (HSA) stage of the processing, white noise burst of short duration or one of 10 novel sound clips of short duration are added to the second audio signal at random time points across the second audio signal to generate the final output audio. This stage is designed to habituate auditory cortex to novel transient sounds. The 10 novel sound clips are sounds of baby crying, fireworks, explosion, glass breaking, thunder, car crash, fire alarm, siren, tire squealing, toilet flushing. For white noise, bursts are 200 milliseconds long, randomly assigned to right, left, or both ears, with a random inter-noise-interval of 10-20 seconds. For novel sounds, each sound clip is 500 milliseconds long, randomly assigned to right, left, or both ears, with a random inter-noise-interval of 10-20 seconds. When both white noise and novel sounds are active, a given 20-second-long segment of audio can have either white noise or novel sound, but not both.

The filters used in NC and AS steps of processing are Infinite Impulse Response (IIR) Filters. An IIR filter is a type of digital filter. The advantage of an IIR Filter is that it is computationally efficient and require little memory. The IIR filter is designed to be stable and have a sharp transition zone.

The details of each of the three processing steps as well as the exemplary presentation of the combination of processing step based on session number will be apparent from the figures and detailed descriptions of the figures.

### BRIEF DESCRIPTION OF THE SEVERAL VIEWS OF THE DRAWINGS

The invention will be better understood with the aid of the following drawings. Additionally, same reference numerals in the drawings designate corresponding parts through several figures. The drawings are:

FIG. 1A is a block diagram that shows an exemplary implementation of the method in the invention.

FIG. 1B is a flowchart of the processing logic and processing steps used to modify the audio signals for therapy of sound hypersensitivity.

FIG. 2A shows the gain-magnitude frequency response of low pass filter used according to the invention.

FIG. 2B shows the gain-magnitude response of high pass filter used according to the invention.

FIG. 2C shows the gain-magnitude response of band stop filter used according to the invention.

FIG. 3 shows the filter specification for band stop filters used for Auditory Shaping.

FIG. 4 shows an exemplary timeline and presentation of the combination of processing steps according to the methods of this invention that is suitable for therapy of sound hypersensitivity in an individual.

FIG. 5 is a flow diagram that shows detailed implementation of Neuroplasticity Conditioning (NC) used according to the invention.

FIG. 6 is a flow diagram that shows detailed implementation of Auditory Shaping (AS) used according to the invention.

FIG. 7 is a flow diagram that shows detailed implementation of Habituation Sound Addition (HSA) used according to the invention.

FIG. 8 shows an exemplary implementation of a real time implementation of the methods according to the invention when the entire input audio signal is locally available on the data processing device.

FIG. 9A shows the exemplary implementation of calculation of loudness levels of a single music file (audio data) when the audio data is a streaming audio signal and is available in temporally consecutive segments.

FIG. 9B shows an exemplary implementation of a real time implementation of the methods according to the invention when the input audio signal is a streaming audio signal and is available in temporally consecutive segments for processing.

### DETAILED DESCRIPTION OF THE INVENTION

FIG. 1A shows the block diagram with the components necessary for an exemplary implementation of method in this invention. This invention uses audio data which are commonly available in digital file format (such as mp3, aac, way, flac). As described in the background, this invention uses music files with vocals since they are soothing for a user to listen to on a daily basis which makes it suitable for purposes of therapy of sound hypersensitivity. The digital music files are available from an external storage server 101. The audio files are then transferred to the local computing device 103 (such as a smartphone or tablet) via the internet 102. The local computing device 103 is a mobile smartphone or a mobile tablet device which acts a data storage device 104 for the music files. The entire catalogue of digital audio files can be transferred via the internet 102 to the local device for storage. In this case, the entire file is available on the local device 103 for processing. Alternatively, the digital file can also be transferred in 102 in small packets of data to the local device 103 when the music is streamed from an external third-party streaming provider (such as Apple Music or Spotify). In addition, the device 103 is also used as the data processing device which modifies the audio files locally for purposes of therapy in 105. Finally, when the files are processed, this device 103 also acts as the audio playback device. The processed output files are then played through the external headphones or earphones 106 connected to the local device.

Therapeutic benefits are expected after listening to music over 40 half hour sessions. For purposes of therapy, the processing of the input audio file is based on the session number to which the audio file belongs to. FIG. 1B shows the detailed implementation of logic and processing steps in this invention based on the session number of the audio song. FIG. 1B represents block 105 in FIG. 1A. The three signal processing steps described in this invention are: (1) Neuroplasticity Conditioning (NC) 1054, (2) Auditory Shaping (AS) 1056, and (3) Habituation Sound Addition (HSA) 1057. An input audio signal 1051 is provided along with a session number 1052 that has a value between 1 and 40 (including 1 and 40). The session number is checked 1053 to see if it belongs to the first five sessions (Sessions 1 to 5) or the last five sessions (Sessions 36 to 40). If the session belongs to the first five or last session sessions, none of the three processing steps are applied to the input audio signal. For session numbers 6 to 35, the input audio is processed with NC 1054. If the audio file belongs to session numbers 11 to 25 (1055), the input audio 1051 processed with NC 1054 is subjected to AS 1056. For audio files belonging to session 26 to 35, the output audio processed with NC 1054 is subjected to HSA 1057. Specifics of the three audio processing steps NC 1054, AS 1056 and HSA 1057 are described in detail later in the detailed descriptions for FIG. 5, FIG. 6 and FIG. 7 respectively. Finally, the magnitude of the audio is scaled to a pre-set audio loudness level 1058 to generate the output audio 106. The pre-set loudness level for songs in each session are listed in FIG. 4.

FIG. 2A, FIG. 2B and FIG. 2C show the gain-frequency response of the three filter types used in processing steps NC and AS with the aid of characteristic curves 201, 202 and 203. The gain-frequency response shows the gain, that is the ratio of the output signal to its input signal versus the frequency over which the filter is operational. Gain is shown on the vertical axis and the frequency is on the horizontal axis. Gain is expressed in decibel scale (dB), where 0 dB is when output signal is equal to the input signal. The −3 dB gain in FIG. 2A, FIG. 2B and FIG. 2C represents the gain when the output signal is 0.707 times the input signal. The frequency at which the gain is −3 dB is referred to as the cutoff frequency of the filter. FIG. 2A shows the characteristic curve 201 of a low pass filter which has a cutoff frequency LF1. The frequencies in the input audio above LF1 that lie in the stop band are blocked by the low pass filter, while frequencies in the audio signal below LF1 in the pass band are unchanged. FIG. 2B shows the characteristic curve 202 of a high pass filter which has a cutoff frequency HF1. The frequencies in the input audio below HF1 that lie in the stop band are blocked by the high pass filter, while frequencies in the audio signal above HF1 in the pass band are unchanged. FIG. 2C shows the characteristic curve 203 of a band stop filter which has a lower cutoff frequency F1 and upper cutoff frequency F2. Frequencies of the input audio signal in the stop band between F1 and F2 are blocked by the band stop filter, while frequencies in the input audio signal below F1 and above F2 are unchanged by the band stop filter. These filters are Infinite Impulse Response (IIR) Filters. IIR filter is a type of digital filter. The IIR filter is designed as a Butterworth filter which provides a maximally flat response (no amplitude ripples in the output signal) in the passband. The width of the transition between the stop band and passband is dictated by the implementation of the digital filter. The filter specifications (dictated by the order of the IIR filter) are chosen for stability as well to achieve a steep transition zone (smaller width of the transition region between the pass band and the stop band).

FIG. 3 gives the specifications of the 17 band stop filters used for Auditory Shaping. The audio signal has a frequency spectrum between ˜20 Hz and ˜20000 Hz. This spectrum can be divided into 11 octave bands, where an octave band is a band where the upper frequency is twice the lower frequency. The upper table is FIG. 3 gives the upper and lower cutoff frequencies for 12 narrow band-stop filters (11 octave filters with an additional filter F10 centered at 5500 Hz). The lower and upper cutoff frequencies for additional band stop filters (F13 to F17) are listed in the lower table in FIG. 3.

The block diagram in FIG. 1B is represented in tabular format in FIG. 4. FIG. 4 shows in a tabular form specific combination of the three processing stages applied to the input audio signal based on session number. For therapeutics benefits, music is presented over 40 half hour sessions with different processing steps for each session. The session numbers are given in column 1 of FIG. 4. As described in the summary, each session or playlist consists of unique songs with a total duration of 30 minutes. Processing is enabled in multiple stages, where each stage consists of 5 half hour sessions, administered as 2 sessions per day with 3+ hours separating each session. A two day ‘rest’ period separates every 10 sessions. Different music is used in each session within a stage, but the same core music (albeit with additional processing) is used in each stage. Thus, 5 unique playlists of songs (each of duration 30 minutes) are repeated every 5 sessions. Playlist named ‘A’, ‘B’, ‘C’, ‘D’ and ‘E’ in column 2 of FIG. 4 represent the 5 unique playlists repeated every 5 sessions. The loudness level for songs in each session are given by column 3 in FIG. 4. The 100% loudness level corresponds to a Loudness Unit Full Scale (LUFS) of −23 LUFS. Loudness Unit Full Scale is a loudness standard designed to enable normalization of audio levels and is defined in ITU BS.1770. The ITU BS.1770 loudness standard describes an algorithm for measuring loudness that manufacturers can use to create loudness meters. When the loudness values are represented by three numbers (for example 60-70-80% in Column 3 of FIG. 4), the first song in a playlist is played at 60% of max loudness, second song in a playlist is played at 70% and the rest of the songs are played at 80% of maximum loudness. The activation state of the NC and AS processing is given by columns 4 and 5 in FIG. 4. Column 6 and 7 in FIG. 4 give the activation state for the two types of HSA (white noise bursts and novel sounds) step of the audio signal processing respectively. The first stage (session number 1 to 5) involves listening to unprocessed broadspectrum music (NC, AS and HSA is turned off), to gain familiarity with the specific music that is subsequently modified. During this stage, the loudness levels are increased from session 1 to session 5, in an effort to de-sensitize the brain to louder sounds. In the next stage (session numbers 6 to 10), music is processed with NC to intentionally produce a very abnormal pattern of cortical stimulation. For the next 3 stages (Session 11 to 25), Auditory Shaping is applied in addition to Neuroplasticity Conditioning. Audio filters used for Auditory shaping were described earlier in the description for FIG. 3. When AS is turned on, the filters for Auditory Shaping listed in FIG. 4 show the exemplary implantation when UCLs are unavailable. If individual UCLs are available, filters for Auditory Shaping are picked from the 12 narrow band filters in FIG. 3 (upper table of octave band filters) based on UCLs. Habituation sound (only white noise bursts) are added to NC in the next stage (Session 26 to 30). Habituations sound which includes a combination of white noise bursts and novel sounds are added to NC for sessions 31 to 35. The final stage (Sessions 36 to 40) involves an ‘auditory reset’, during which the original unmodulated music is re-presented (now at 80% of max loudness), with the goal of allowing the auditory system to ‘settle’ into a normal perceptual mode.

FIG. 5 shows the flowchart for the detailed implementation of the Neuroplasticity conditioning step 1054 used in FIG. 1B. Audio signal parameters necessary for analysis are first extracted in 501 from the input audio signal. The sampling frequency (Fs) and the duration of the audio signal in seconds (L) is extracted from the input audio signal. The signal is divided into ‘N’ temporally consecutive non-overlapping segments where each segment is 0.5 seconds long where N equals the total number of 0.5 second segments in the audio signal. Based on Fs from 501, three digital filters are designed 502 to block out frequencies from the audio signal in the low frequency band (0 to 500 Hz), mid-frequency (500 Hz to 4000 Hz) and high frequency band (4000 Hz to 20000 Hz) respectively. Ideally, this would be achieved with a high pass filter with cutoff frequency of 500 Hz, band stop filter with cutoff frequencies at 500 Hz and 4000 Hz and a low pass filter with a cutoff frequency at 4000 Hz. To achieve a higher reduction of amplitudes at the cutoff frequencies, the three filter are designed with a modified cutoff frequency in the invention. For example, the Type 1 filter (high pass filter) with a cutoff frequency of 500 Hz causes a reduction in amplitude of the output signal by a factor of 0.707 at the cutoff point. To achieve a reduction of amplitude at 500 Hz, the cutoff frequency for the Type 1 filter for NS is set at 800 Hz. Similar modifications are made to the Type 2 and 3 filters for NS. Thus, the three NS filters in the invention are: Type 1 is high pass filter with cutoff frequency at 800 Hz (0-800 Hz is blocked off by Type 1), Type 2 is a band stop filter with lower and upper cutoff frequencies of 300 Hz and 5500 Hz respectively (300-5500 Hz is blocked by Type 2), Type 3 is a low pass filter with cutoff frequency of 3775 Hz (frequencies from 3775 to Fs/2 Hz are blocked Type 3). In the next step 503, filters are assigned for each of the N segments of the audio signal. Type 1 filter is assigned to the first 20% of the N segments, Type 2 filter is assigned to 20% of N segments and Type 3 filter is assigned to the 50% of N segments. No filter is assigned for the remainder of N segments (10% of N) and those segments are unprocessed by NC. The list of filter types assigned to the N segments are then shuffled to randomize the assignment of filter type to each of the N segments in 504. For a 2-channel audio signal 505, steps in block 503 are repeated for the second channel to generate the filter list for N segments of the second channel. 50% of the filter types calculated for the second audio signal (channel) are then replaced by the value calculated to the second audio signal (channel). The final list of filter types calculated for the second channel are finally shuffled. Filters are finally applied to each of the N segments in both channels according to the filter list generated for each channel in 506. The steps in 503, 504 and 505 thus helps in a generation of a randomized pattern of filtering in three frequency bands for each channel in the audio signal as well across the two channels of the audio signal.

FIG. 6 is a flow diagram that shows detailed implementation of Auditory Shaping (AS) step 1056 used in FIG. 1B. Availability of an individual's UCLs is first checked in 601. If UCLs are available 602, pick from one of 12 octave filters in FIG. 3 based on the frequencies that are uncomfortable for the individual. For the case when UCLs are unavailable, filters (from FIG. 3) for Auditory Shaping are selected based on the session number the audio file belongs to as described in FIG. 4.

FIG. 7 is a flow diagram that shows detailed implementation of Habituation Sound Addition (HSA) 1057 used in FIG. 1B. This step adds a short duration audio signal (200 milliseconds to 500 milliseconds long) to the audio processed by NS stage at random times in the input audio signal. The audio segments to be inserted is one of two types: (1) white Noise burst 200 milliseconds long (2) random selection from a white Noise burst of duration 200 ms and 10 other novel sounds where each sound novel sound is 500 milliseconds long (baby crying, fireworks, explosion, glass breaking, thunder, car crash, fire alarm, siren, tire squealing, toilet flushing). The sound segments (stimulus) are randomly assigned to right, left, or both ears, with a random interstimulus-interval of 5-15 seconds. Individuals with sound hypersensitivities are generally uncomfortable with these noise bursts and novel sounds and the idea is to habituate the sound sensitive individual to these sounds. The total duration of the input audio signal, the habitation sound type, and session number are the required input parameters for HSA 701. The next steps of processing are based on the habituation sound type 702 that is active for a particular session number (see FIG. 4 correspondence between session number and habituation sound type). The stimuli (habituation sound) is added to the input audio signal with a varying inter-stimulus-interval of 5 seconds to 15 seconds. For the case when the habituation sound is purely white noise 703, a list of time points at which the sounds will be inserted is generated with randomized inter-noise-interval of 5 to 15 seconds. When the habituation sound consists of both white noise and novel sounds 704, two independent list of time points (one for white noise and the other for novel sounds) at which the habituation sound clips will be inserted is generated with randomized ISI of 5 to 15 seconds. Following that, insertion timings are randomly chosen from one of the two lists for every consecutive 20 second block for the duration of the input audio signal 705. This ensures that every 20 second segment of the input audio contains either white noise or novel sounds and does not contain both. In addition, every novel sound segment added to the input signal is randomly selected from the list of 11 pre-defined audio signals 707. Each habituation sound segment is then randomly assigned to first channel, second channel or to both channels for a 2-channel audio signal for white noise 706 or for a combination of white noise and novel sounds 707. The habituation audio segment is then added to the audio signal processed by NC based on the previously generated time point insertion list and channel assignment from 708.

FIG. 8 shows an exemplary implementation of a real time implementation of the methods according to the invention when the entire input audio signal (digital music file) is locally available on the data processing device. The data processing device 103 contains the computer code for the data processing methods described in this invention and also acts as the audio data storage and the audio playback device. The audio files to be processed are first transferred from an external storage 101 to the data processing device 103 via the internet 102. Thus, the entire digital audio files required for all sessions are now available on the data processing device 103. The external storage 101 can be an external hard drive or an external server with storage space. The audio files on the external server 101 are loudness normalized to a pre-set level of −23 LUFS so that the user is not subjected to changes in audio loudness between songs. The data processing device 103 also acts a data storage device 104 for the music files. In addition, the data processing device 103 contains the computer code for audio processing and a text base database 804 (for example an XML—Extensible Markup Language data file) that contains audio parameters for processing the audio songs (audio data) required for purposes of therapy. The tabular data in FIG. 4 is embedded in the text based database 804 and contains parameters for each song such as the song name, song artist name, song duration, session number that the song belongs to, sampling frequency of digital audio data, playback loudness level, the processing stages to be applied to the song (NC, AS and HSA) and the parameters of the 3 processing stages (NC, AS and HSA). In 805, the NC filter list, AS filter list and the list of novel sound insertion timings is generated as described in FIG. 5, FIG. 6 and FIG. 7 respectively using the audio file parameters saved in the text-based database file 804. The digital filter type to be applied (for NC and AS) and the habituation sounds to be inserted (for HSA) are calculated for consecutive and non-overlapping 0.5 second segments of the input audio song in 805. This pre-processing step in 805 allows for real time processing of the input audio signal. The input audio file is opened for processing 806 and the digital audio samples are read into an input memory buffer 806. The session number that the song belongs to, loudness level for the song and the processing stages (NC, AS and HSA) to be applied (as described in both FIG. 1B and FIG. 4) are available from the text-based database file 804. In 105, the selected processing steps are applied to the 0.5 seconds of audio data and finally the magnitude of the audio is scaled to generate a pre-set audio loudness level for playback. The processed data is then directly sent to the device audio buffer for playback 808 through a headphone or earphone 106. If the end of the audio file is not detected 810, the next block of audio data containing 0.5 seconds of data is read 806 and processed as previously done in 105 and sent to the output device buffer 808 for playback through a headphone or earphone 106. Processing of the audio data is stopped when the end of the audio file is detected 809.

In the case of streaming audio, the entire audio file is not available, and the audio data is sent/streamed via the internet to the device in segments until the end of the file is reached. In such scenario, the loudness level of each song may not be available, hence processing of the streaming audio data is required to calculate the loudness levels for normalization of songs used for therapy of sound hypersensitivity. FIG. 9A shows the exemplary implementation of calculation of loudness levels of a single music file (audio data) when the audio data is a streaming audio signal and is transmitted to the device in temporally consecutive segments. FIG. 9A is a pre-processing stage to for streaming audio. The audio files are available from an external storage server 101 and data in each file is streamed to the data processing device 103 over the internet 102 by a streaming audio service (such as Spotify or Apple Music). Streaming of the audio file is started in 904 and the transmitted audio data is saved to a circular ring buffer 905 that has the capacity to store 30 seconds of audio data. A circular buffer is a commonly used data structure (more description can be found on Wikipedia) that uses a single, fixed-size memory buffer as if it were connected end-to-end. Circular buffers stores data on a first-in, first-out basis and are more memory efficient. When the buffer is completely full 906, the streaming is paused 907. 0.5 seconds of audio data is read from the circular ring buffer 908, the loudness level (LUFS) for this block is calculated as per ITU specs (standardized in ITU-R BS.1770) and saved to a temporary memory 9081, and the audio data is then removed from the ring buffer 911. The read 908, loudness calculation 9081 and deletion 911 from circular buffer is continued until the entire data in circular ring buffer is processed. At the same time, when the data in the circular buffer drops below 30 seconds worth of data 914, the streaming of audio data is resumed 904. The circular buffer is checked to find out if it is empty 912. When the circular ring buffer is completely empty, a check is run to find out if streaming has reached the end of the audio file 913. If the end of file has not been reached, streaming is resumed 904. If the end of the file has been reached, the overall loudness of the file is calculated 915 by averaging the loudness values of all non-overlapping 0.5 second segment of audio data calculated in 9081 as per ITU specs (standardized in ITU-R BS.1770). The loudness value is stored on a file on device 103.

After the loudness level calculation of streaming audio is done as per FIG. 9A, the audio file is processed for therapy of sound hypersensitivity. FIG. 9B shows an exemplary implementation of a real time implementation of the methods according to the invention when the input audio signal is a streaming audio signal and is available in short and temporally consecutive segments for processing. Similar to FIG. 8, the data processing device 103 in FIG. 9B contains the computer code for the streaming audio data processing methods described in this invention and also acts as the audio data storage and the audio playback device. Blocks 804 and 805 described in FIG. 8 are the next steps in FIG. 9B. A text base database (804) that contains audio parameters for the list of audio songs (audio data) required for purposes of therapy is available along with the computer code for processing. Using the audio file parameters saved in the text-based database file 804, the NC filter list, AS filter list and the list of novel sound insertion timings is generated as described in FIG. 5, FIG. 6 and FIG. 7 respectively. Streaming of the audio file is started in 904 and the transmitted audio data is then saved to a circular ring buffer 905 that can hold data of 30 second duration. When the buffer is completely full 906, the streaming is paused 907. Streaming is continued until the buffer is filled or the end of the file is reached 9061. The session number that the song belongs to and the processing stages (NC, AS and HSA) to be applied (as described in FIG. 1) are available from the text-based database file 804. The loudness levels for the audio file is also available from the text-based database 804. 0.5 seconds of data is now read from the circular ring buffer 908 and in 105, the selected processing steps and loudness audio scaling is applied to the 0.5 seconds of audio data. The processed data is then directly sent to the device audio buffer for playback 910 and then played through a headphone or earphone 106. The processed data buffer is then removed from the ring buffer 911. The read 908, process 105 and deletion 911 steps from the circular buffer is continued until the entire data in circular ring buffer is processed and removed. At the same time, if the data in the circular buffer drops below 30 seconds worth of data 914, the streaming of audio data is resumed 904. The circular buffer is checked to find out if it is empty 912. When the circular ring buffer is completely empty, a check is run to find out if streaming has reached the end of the audio file 913. If the end of file has not been reached, streaming is resumed 904.

What is claimed is:

1. A method for processing audio signals in a manner that leads to a reduction of sound hypersensitivity, with the sound modulation profile based on an individual's UCLs, the method comprising of:
provision of an audio signal (104) with two audio channels;
subjecting the original audio signal to high-pass, mid-frequency band-stop and a low-pass filtering randomly over 0.5 seconds of temporally consecutive segments, and applied separately and randomly to the two audio channels (1054) to generate a second audio signal; and
subjecting the second audio signal to additional band stop filters which are individually set based on psychophysical evaluation of uncomfortable loudness levels (1056) to generate a third audio signal or adding short duration novel sounds at random temporal locations (1057) in the second audio signal to generate a third audio signal.

2. The presentation of the audio processed by the method in claim 1 over a specific duration (40 total sessions which consists of two 30-minute sessions per day, 5 days per week for 4 weeks) for therapy of sound hypersensitivity.

3. The method for application of a specific combination (105) of the method in claim 1 based on the session number that the audio signal (104) belongs to, where the session number ranges from 1 to 40 as per claim 2 for reduction of sound hypersensitivity.

4. The method according to claim 1 where the second audio signal is subjected to a pseudo-random selection of band stop filters when the individual's uncomfortable loudness levels are unavailable (603).

5. The method according to claim 1 where the short duration novel sounds (1057) are 200 millisecond white noise burst or one of the following 500 millisecond audio signals: baby crying, fireworks, explosion, glass breaking, thunder, car crash, fire alarm, siren, tire squealing and toilet flushing.

6. The method according to claim 1 where the filters used are infinite impulse response filters.

7. The method according to claim 1, wherein the method is performed using a data processing device (103).

8. A computer program product where a computer code executes the method in claim 1 on a data processing device (103).

9. The method according to claim 1 where in the first audio signal is a digital audio signal, in particular a digital audio file.

10. The method according to claim 3, wherein the audio is processed with a specific combination of method in claim 1 based on the session number of the audio signal, the method comprising of:
provision of an audio signal (104) with two channels along with a session number;
the audio signal is unprocessed when the session number is between 1 to 5 (including session number 1 and 5) or between 35 to 40 (including session number 35 and 40);
subjecting the first audio signal to high-pass, mid-frequency band-stop and a low-pass filtering randomly over 0.5 seconds of temporally consecutive segments, and applied separately and randomly to right and left ears (1054) to generate a second audio signal when the session number of first audio signal is between 6 to 10 (including session number 6 and 10);
subjecting the first audio signal to high-pass, mid-frequency band-stop and a low-pass filtering randomly over 0.5 seconds of temporally consecutive segments, and applied separately and randomly to right and left ears (1054) to generate a second audio signal, followed by subjecting the second audio signal to additional band stop filters (1056) which are individually set based on psychophysical evaluation of uncomfortable loudness levels (602) to generate a third audio signal when the session number of first audio signal is between 11 to 25 (including session number 11 and 25);
subjecting the first audio signal to high-pass, mid-frequency band-stop and a low-pass filtering randomly over 0.5 seconds of temporally consecutive segments, and applied separately and randomly to right and left ears (1054) to generate a second audio signal, followed by adding a short duration white noise burst at random temporal locations (1057) to the second audio signal to generate a third audio signal when the session number of first audio signal is between 26 to 30 (including session number 26 and 30); and
subjecting the first audio signal to high-pass, mid-frequency band-stop and a low-pass filtering randomly over 0.5 seconds of temporally consecutive segments, and applied separately and randomly to right and left ears (1054) to generate a second audio signal, followed by adding a short duration white noise burst or novel sounds at random temporal locations (1057) to the second audio signal to generate a third audio signal when the session number of first audio signal is between 31 to 35 (including session number 31 and 35).

11. The method according to claim 10 where the first audio signal is subjected to high-pass, mid-frequency band-stop and a low-pass filtering randomly over 0.5 seconds of temporally consecutive segments, and applied separately and randomly to right and left ears (1054) to generate a second audio signal followed by subjecting the second audio signal to additional pseudo random selection of band stop filters (1056) when the individual's uncomfortable loudness levels are unavailable (603) to generate a third audio signal when the session number of first audio signal is between 11 to 25 (including session number 11 and 25).
